SEQUENCE LISTING (30 sequences)

SEQ ID NO: 1 - DNA, 39 bp, Artificial Sequence
  This sequence is synthesized in lab.
gttgacatgcctaataaaagatttggttggatgaatttc

SEQ ID NO: 2 - DNA, 381 bp, Artificial Sequence
  This sequence is synthesized in lab.
cttaatttccagtacattttcattgcccattgagatcatcactggcttttcaaaaggcttaaacacaggattatgtacataaatgagatcaaagtatttggaaaggatcccatctaacatgaaaaaggctttggttttcagggggagttgcagattcagctgttgcagggaaggagtggtacaacagattatctctgaattagagcgatgttgacatgcctaataaaagatttggttggatgaatttcatagacaatgggatcttcacggtaactgaagatgcttgtctctcggttggctaagtcaattttcaatttaacagcaaactcagttgaaatggtttgggctggggtataacattcaagaatactgtttgacaca

SEQ ID NO: 3 - DNA, 6537 bp, Artificial Sequence
  This sequence is synthesized in lab.
tacaagatgttgcatcactttactttaattgcatgatttatcagaacaactattaacatacgaagtaccattcagttcagctgcaggtataggcagtgacaagtatctaattcttagaagaatcacttactcccacaatctgtccagacacattaatctaaggacaagtttataaatagcaaacgtgattttcacattgcagtgttctcaagaatgtatatacaagtgtgtagtcctgttgatgggatgtttccccgagttctttctattgatgcgttcatgctcttgaccctggtagagacagttctttctttccacagagcagattttcttttgtcatccaccatttacaatactctgtgaattagtattacttattacatttatgaaacggcaatatttggattgtaatagctcttcagtacaattccttgtgtcttctggtaagtctctgcactatcagagagcttttagttataatcattttcctgcaacaacagccaaactcaactattgagtttcagtgtgacacaccctctttgtagcttgctgggttaacccttggcttcaagtcctgatgatgtaatgagggtggggtgtattggcaatcagtacatttccttatcgcaatttacagtcattgaaaatcatgctgtcattaatcccagtctgacataccttttctaaaatgttcacagtgcagtgtttttgtggcctaacaaaatttttctcatatcattaaaaataaacatttttataaaaaatataacactttaaatgtttacgtcgacaaaaccagttagagtaacctacaccacatgcactatacagtagcaagcacaaaattccacagaatgaagcatcacaaagttctgctcagggtggctattccatctaggtgaaatagctgggattttcaattgcctttttcatttgtttctaaagtatgttttgcttaacataaaacacaccctaatgcaaaataaaactccccaaaagttttgtttccaattgcttgcgaggtgggaacctgccaccgagacagaggctaatcttttcaatccatccaccctttctttgctctacctatgagctgtgattggaaccaatgaaccttttagtaaaatgtatcctgctttacaaacatgctgagttatctttaaaaatatttatcaacaaattacttgtcttattttgagttttcatttaaaaaaatacacacaaaacatctacatgttcacattcattagatcagagtagcatcattctcaaacagtgggtttttatatgacaactaaatatttcatgatgactaaattatttcatacaaattttcttaatgtcatctatgtacattacatcctgttcatttataaacatgttttgtgtgatgctattttgaaactttgactgtattgcctataaatattcttccaaccaaaggaatgtctatacaaaaatttataagggctgggcgcagtggctcatgcctgtaatcctagcactttgggaggctgaggtgggtggatttcttgagtccaggagtttgaaaccagccttggcaacatggcaaaaccccgtctctacaaaaaatacaaaaattagccggggatggtggtgtgcgcctgtagtcccagctattcgggaggcttgagcctgggaggtggaggttgcagtgagctatgatcacaccactgcactacagccctggcaacagagtgagatcctgtctcaaaaaacaaacaaacatttaaaagggaatgtttacctaatgggtgaatgacaccatcagtaatgaaaaatgttattgaccatacaacacacaaaagtgttttgaaacactagaattctaaatgagtggcctgttctggggctgccgctcctgtcctgagcattactatctcttctgagttttctgtgatcaagaagccctcaatatttcctgcttcaatttcccatatgaaatcaagtggtacctgatttttaaaaaattcaacccagaattcaagttttggactccaatatgacaggagtgttgtcacggctgcaggccattggtccgtggcctgtgggaccaagcctctggttctgatgctctgtcagataagaaattccttagaatccagtaatttaaataacaatacaagtcctataatagtgcaattttggcaagagcaaagaatatcgatggccttttaaaggtcaggcagtgaaaaaaccattggacaaagtgtggactgttgctttgacatagtactagcactatgatgtctcccagaaggaggctggtcgtgtgtccacctcatcatcagcgttatcttctgatgacaacagagaaggatacggagcgacacattttacgttcacataagtagcgttcacatggacatagtgctccccaatgaaagtagagaagatcgctgatatccgggacaccagttcagaaaaggatgggcgcatttcggctttagggtgccagcattttagcattacttcatataaggggtctgggcagtattcgggttgtaggagtcttctcccttgcaacaagtaaacagttatatcaaaggtgtttacgtcaggataaggtggggctcctcttgtcatcagctcccagaggagcacgccaaaggaccacacatctgacttggtggtaaacttttgagtttgcagactttccaaagccatccacttcactggcagctttgcacctgttttgttgtgtacactatagtattctttatcatacatgtctctggcaagaccaaaatcagcaaccttgactgtgaatttttcatccagcatacagtttcttgcagccaagtctctgtggacaaactttttgcttgcaagatatttcatgcctttggctacttgaagaccaaagccaataagatcttttacagttggattatgagtctcatttcgaatgaaatttcgaagatctccatgtttcatgtatggtaggaccaccagcggagacccttcacttcgcaggcagattcccaggagcgagaggacattgggatgactaaaatctttcatgatgattccctcggtcagaaattgggaaacttctcctatgtcagtgattctgttcaaggatttcacagcacagtgaattttcttgccatcattgtccaacaaagtcccatgatatacacaaccaaaatgccctcttcctatgacttcattgaaatgcacaatcaggctactgggcccaatcactacatgctgcactgcctggaccagctctggatttagagcactgaggtcaatgtggacagtattttgcagtaatggactggatatatcagagtccccactagttaggatgggggacatgtctgtcagaggatactgcacttgtcggcatgaaccgttctgagatgaattaggaaactgatcttctggaaaagtagctcggtagtctacagattcatttgaaaccatttctgtagttgggcttacacttcgggcacttacaagcctatccaaatgaggagtgtgtactcttgcatcgtagcgaactaattcactgcccagatctttaatttgctttctctttttcagccacaggaaaaacccaagtagtaataacagtgctgttgatattgagacaacaccagcaatcaatcctgtgaaattctgatctggttgaactattacttttccaaggacggttgaagaaattgcttgcttccactctatatttagctcgctgttcaatttcagcaggtcattggggaccgtgcataaaacggcttcagaatgtaagtgtatattctcacagctcttatttccaacttttaacacttcacctttaactgcttcagggtcaatatcatttcccttaatttccagtacattttcattgcccattgagatcatcactggcttttcaaaaggcttaaacacaggattatgtacataaatgagatcaaagtatttggaaaggatcccatctaacatgaaaaaggctttggttttcagggggagttgcagattcagctgttgcagggaaggagtggtacaacagattatctctgaattagagcgatgttgacatgcctaataaaagatttggttggatgaatttcatagacaatgggatcttcacggtaactgaagatgcttgtctctcggttggctaagtcaattttcaatttaacagcaaactcagttgaaatggtttgggctggggtataacattcaagaatactgtttgacacactttttaaagtacatgtttttccaccaattgaaatgtgtctagaattcccactgtttaggtaatttccagttaaagtaagtaaagtgccaccagccataggaccgtatttcggcgaaatacttgttattacaggatccacataggagaatgtactgtattgtgttgtcccgtggccatttgaaataattatggacatattgaaatgcttattcatggcaggaccaactgtgcatttcaatgtattcatcgtgctctcacttaaagtcaaggtgcagctctcatttccaaggagaactctagttttctttaaatcaaatttattattcctccgaaatccaaagtcccagccacatatggtcagccttgtccctccttcaaggggtgcactatttgggaaaaccttgtagattgcaggcagacagatctgttgagtccatgtcccgctcaggcattcctccgatcgcacacatttgtcgtggcaccagccacactgaacaaagggtggggcagagaggcattgactgcaggactggaaatgtctgcagcccaagccattcaatgggatcttcgtgatcttcttcccagtgataaccagtgtgtagccattttggtttaatgtatgctccacaatcacttctggagacactggatgggagtccaggagaaaattcacatgaggggttgatggtcctgatcgagaaaccacaacctgcatgaagcgaccctctgatgtcccaagattagctatggtgaggtctcctttaatgaaggtggatatagatgttaagaggacttcgctgaattgacccatgaataagtcaacgcgctgcaaagctgtggtaaactctgttcgatattcatcacggcgcgcttcacagcctgatgaatttctcagaagtgtcctattaaagcagtgctcatgattgggtccgtaaaaatgctggagacatctcacattgtttttgttgacgatcttgttgaagaagtcgttgacatatttgatagggaatgcacacatggcagatcgatccattggttcggcagaatctggcttgctttgtgcgaacaccccgaaaagaatgtcatcattcaggctggctcctatttgtctagcaagctgggccccaggcttgctgacatacgcagcctgaagtatattaaacacttccttctttgtggatctcttttttctcttttctgtgagaatacactccagaggcatttccatgtaggaatgcaatccagagtttatggaacagaacctgattattcttgtgtgaaaagtctgagcatctagagtttccctttggaccgtcaagaagtaaataaaattgttgctttcaaaggcatggacatacttaatggggtaagaatctctgaactcaggtaaaacatcaatgtaggactggtccgtcaaaaacataaaaccatctttcgtttcctttagccttctcactgatatcgaatgcaatggatgatctgggaaataagaagaatttatggtattgcctacaaagaagttgatgaaccggtcctttacagatgaaaggactttggctcccagggcgctcaccacacagtcaggacactggctgggctcttctatctgtggggagaatatgcagtgaacctccgactgtatgtcagcagtatgattgtggggaaagacatgtcgctggcaggtccctctgttgacgctgccacagctaatgagttgatcatcatagtaggtgtcgacaactagagccatgttgatgttatctttccaaacacctcctgataaattggctttgctgctgcagtcctgacatgggaaacaatctgggtgttccagcacaggcccagtcttgtactcagcaaccttctgaaggtcttcctcatttaaaacataaatgtagttagtggcaccaaggaaaatgtgatgctcatgtagaatgacattctggatgggtgtttccgcggtgaagttgggaagctgatacttcatattcacattcatctcggactttgctagtgcctctttacactccccattgctcctctgcaccaaggtaaacaggagcacgaggatgccaggtgcaagcacagcgggggccttcattatgagaggtttatctttcggtgcccaggaaccagtggagaagtcagcggcgcaaggaccacacgcgcgctccgcgcctccccgcctcctctcagcaagtcagctgtcgccccgcatctggctcgcgccctccactcggctccgcatctgctcacaaagcgctcggggcgccgcgggcggcgagggcctccgggtcacctgc

SEQ ID NO: 4 - DNA, 60 bp, Artificial Sequence
  This sequence is synthesized in lab.
tcagctcccagaggagcacgccaaaggaccaatacagtttcttgcagccaagtctctgtg

SEQ ID NO: 5 - DNA, 247 bp, Artificial Sequence
  This sequence is synthesized in lab.
aaggggtctgggcagtattcgggttgtaggagtcttctcccttgcaacaagtaaacagttatatcaaaggtgtttacgtcaggataaggtggggctcctcttgtcatcagctcccagaggagcacgccaaaggaccaatacagtttcttgcagccaagtctctgtggacaaactttttgcttgcaagatatttcatgcctttggctacttgaagaccaaagccaataagatcttttacagttggatt

SEQ ID NO: 6 - DNA, 6471 bp, Artificial Sequence
  This sequence is synthesized in lab.
tacaagatgttgcatcactttactttaattgcatgatttatcagaacaactattaacatacgaagtaccattcagttcagctgcaggtataggcagtgacaagtatctaattcttagaagaatcacttactcccacaatctgtccagacacattaatctaaggacaagtttataaatagcaaacgtgattttcacattgcagtgttctcaagaatgtatatacaagtgtgtagtcctgttgatgggatgtttccccgagttctttctattgatgcgttcatgctcttgaccctggtagagacagttctttctttccacagagcagattttcttttgtcatccaccatttacaatactctgtgaattagtattacttattacatttatgaaacggcaatatttggattgtaatagctcttcagtacaattccttgtgtcttctggtaagtctctgcactatcagagagcttttagttataatcattttcctgcaacaacagccaaactcaactattgagtttcagtgtgacacaccctctttgtagcttgctgggttaacccttggcttcaagtcctgatgatgtaatgagggtggggtgtattggcaatcagtacatttccttatcgcaatttacagtcattgaaaatcatgctgtcattaatcccagtctgacataccttttctaaaatgttcacagtgcagtgtttttgtggcctaacaaaatttttctcatatcattaaaaataaacatttttataaaaaatataacactttaaatgtttacgtcgacaaaaccagttagagtaacctacaccacatgcactatacagtagcaagcacaaaattccacagaatgaagcatcacaaagttctgctcagggtggctattccatctaggtgaaatagctgggattttcaattgcctttttcatttgtttctaaagtatgttttgcttaacataaaacacaccctaatgcaaaataaaactccccaaaagttttgtttccaattgcttgcgaggtgggaacctgccaccgagacagaggctaatcttttcaatccatccaccctttctttgctctacctatgagctgtgattggaaccaatgaaccttttagtaaaatgtatcctgctttacaaacatgctgagttatctttaaaaatatttatcaacaaattacttgtcttattttgagttttcatttaaaaaaatacacacaaaacatctacatgttcacattcattagatcagagtagcatcattctcaaacagtgggtttttatatgacaactaaatatttcatgatgactaaattatttcatacaaattttcttaatgtcatctatgtacattacatcctgttcatttataaacatgttttgtgtgatgctattttgaaactttgactgtattgcctataaatattcttccaaccaaaggaatgtctatacaaaaatttataagggctgggcgcagtggctcatgcctgtaatcctagcactttgggaggctgaggtgggtggatttcttgagtccaggagtttgaaaccagccttggcaacatggcaaaaccccgtctctacaaaaaatacaaaaattagccggggatggtggtgtgcgcctgtagtcccagctattcgggaggcttgagcctgggaggtggaggttgcagtgagctatgatcacaccactgcactacagccctggcaacagagtgagatcctgtctcaaaaaacaaacaaacatttaaaagggaatgtttacctaatgggtgaatgacaccatcagtaatgaaaaatgttattgaccatacaacacacaaaagtgttttgaaacactagaattctaaatgagtggcctgttctggggctgccgctcctgtcctgagcattactatctcttctgagttttctgtgatcaagaagccctcaatatttcctgcttcaatttcccatatgaaatcaagtggtacctgatttttaaaaaattcaacccagaattcaagttttggactccaatatgacaggagtgttgtcacggctgcaggccattggtccgtggcctgtgggaccaagcctctggttctgatgctctgtcagataagaaattccttagaatccagtaatttaaataacaatacaagtcctataatagtgcaattttggcaagagcaaagaatatcgatggccttttaaaggtcaggcagtgaaaaaaccattggacaaagtgtggactgttgctttgacatagtactagcactatgatgtctcccagaaggaggctggtcgtgtgtccacctcatcatcagcgttatcttctgatgacaacagagaaggatacggagcgacacattttacgttcacataagtagcgttcacatggacatagtgctccccaatgaaagtagagaagatcgctgatatccgggacaccagttcagaaaaggatgggcgcatttcggctttagggtgccagcattttagcattacttcatataaggggtctgggcagtattcgggttgtaggagtcttctcccttgcaacaagtaaacagttatatcaaaggtgtttacgtcaggataaggtggggctcctcttgtcatcagctcccagaggagcacgccaaaggaccaatacagtttcttgcagccaagtctctgtggacaaactttttgcttgcaagatatttcatgcctttggctacttgaagaccaaagccaataagatcttttacagttggattatgagtctcatttcgaatgaaatttcgaagatctccatgtttcatgtatggtaggaccaccagcggagacccttcacttcgcaggcagattcccaggagcgagaggacattgggatgactaaaatctttcatgatgattccctcggtcagaaattgggaaacttctcctatgtcagtgattctgttcaaggatttcacagcacagtgaattttcttgccatcattgtccaacaaagtcccatgatatacacaaccaaaatgccctcttcctatgacttcattgaaatgcacaatcaggctactgggcccaatcactacatgctgcactgcctggaccagctctggatttagagcactgaggtcaatgtggacagtattttgcagtaatggactggatatatcagagtccccactagttaggatgggggacatgtctgtcagaggatactgcacttgtcggcatgaaccgttctgagatgaattaggaaactgatcttctggaaaagtagctcggtagtctacagattcatttgaaaccatttctgtagttgggcttacacttcgggcacttacaagcctatccaaatgaggagtgtgtactcttgcatcgtagcgaactaattcactgcccagatctttaatttgctttctctttttcagccacaggaaaaacccaagtagtaataacagtgctgttgatattgagacaacaccagcaatcaatcctgtgaaattctgatctggttgaactattacttttccaaggacggttgaagaaattgcttgcttccactctatatttagctcgctgttcaatttcagcaggtcattggggaccgtgcataaaacggcttcagaatgtaagtgtatattctcacagctcttatttccaacttttaacacttcacctttaactgcttcagggtcaatatcatttcccttaatttccagtacattttcattgcccattgagatcatcactggcttttcaaaaggcttaaacacaggattatgtacataaatgagatcaaagtatttggaaaggatcccatctaacatgaaaaaggctttggttttcagggggagttgcagattcagctgttgcagggaaggagtggtacaacagattatctctgaattagagcgatgttgacatgccactgtaaagttccttcctgcttcatgcacatttatgaccattctcgggacactaactgaattcaggtttttcccaacacctgttattgtgctcccaccactaataaaagatttggttggatgaatttcatagacaatgggatcttcacggtaactgaagatgcttgtctctcggttggctaagtcaattttcaatttaacagcaaactcagttgaaatggtttgggctggggtataacattcaagaatactgtttgacacactttttaaagtacatgtttttccaccaattgaaatgtgtctagaattcccactgtttaggtaatttccagttaaagtaagtaaagtgccaccagccataggaccgtatttcggcgaaatacttgttattacaggatccacataggagaatgtactgtattgtgttgtcccgtggccatttgaaataattatggacatattgaaatgcttattcatggcaggaccaactgtgcatttcaatgtattcatcgtgctctcacttaaagtcaaggtgcagctctcatttccaaggagaactctagttttctttaaatcaaatttattattcctccgaaatccaaagtcccagccacatatggtcagccttgtccctccttcaaggggtgcactatttgggaaaaccttgtagattgcaggcagacagatctgttgagtccatgtcccgctcaggcattcctccgatcgcacacatttgtcgtggcaccagccacactgaacaaagggtggggcagagaggcattgactgcaggactggaaatgtctgcagcccaagccattcaatgggatcttcgtgatcttcttcccagtgataaccagtgtgtagccattttggtttaatgtatgctccacaatcacttctggagacactggatgggagtccaggagaaaattcacatgaggggttgatggtcctgatcgagaaaccacaacctgcatgaagcgaccctctgatgtcccaagattagctatggtgaggtctcctttaatgaaggtggatatagatgttaagaggacttcgctgaattgacccatgaataagtcaacgcgctgcaaagctgtggtaaactctgttcgatattcatcacggcgcgcttcacagcctgatgaatttctcagaagtgtcctattaaagcagtgctcatgattgggtccgtaaaaatgctggagacatctcacattgtttttgttgacgatcttgttgaagaagtcgttgacatatttgatagggaatgcacacatggcagatcgatccattggttcggcagaatctggcttgctttgtgcgaacaccccgaaaagaatgtcatcattcaggctggctcctatttgtctagcaagctgggccccaggcttgctgacatacgcagcctgaagtatattaaacacttccttctttgtggatctcttttttctcttttctgtgagaatacactccagaggcatttccatgtaggaatgcaatccagagtttatggaacagaacctgattattcttgtgtgaaaagtctgagcatctagagtttccctttggaccgtcaagaagtaaataaaattgttgctttcaaaggcatggacatacttaatggggtaagaatctctgaactcaggtaaaacatcaatgtaggactggtccgtcaaaaacataaaaccatctttcgtttcctttagccttctcactgatatcgaatgcaatggatgatctgggaaataagaagaatttatggtattgcctacaaagaagttgatgaaccggtcctttacagatgaaaggactttggctcccagggcgctcaccacacagtcaggacactggctgggctcttctatctgtggggagaatatgcagtgaacctccgactgtatgtcagcagtatgattgtggggaaagacatgtcgctggcaggtccctctgttgacgctgccacagctaatgagttgatcatcatagtaggtgtcgacaactagagccatgttgatgttatctttccaaacacctcctgataaattggctttgctgctgcagtcctgacatgggaaacaatctgggtgttccagcacaggcccagtcttgtactcagcaaccttctgaaggtcttcctcatttaaaacataaatgtagttagtggcaccaaggaaaatgtgatgctcatgtagaatgacattctggatgggtgtttccgcggtgaagttgggaagctgatacttcatattcacattcatctcggactttgctagtgcctctttacactccccattgctcctctgcaccaaggtaaacaggagcacgaggatgccaggtgcaagcacagcgggggccttcattatgagaggtttatctttcggtgcccaggaaccagtggagaagtcagcggcgcaaggaccacacgcgcgctccgcgcctccccgcctcctctcagcaagtcagctgtcgccccgcatctggctcgcgccctccactcggctccgcatctgctcacaaagcgctcggggcgccgcgggcggcgagggcctccgggtcacctgc

SEQ ID NO: 7 - RNA, 59 nt, Artificial Sequence
  This sequence is synthesized in lab.
gaaauucauccaaccaaaucuuuuauuaggcaugucaacaucgcucuaauucagagaua

SEQ ID NO: 8 - RNA, 381 nt, Artificial Sequence
  This sequence is synthesized in lab.
ugugucaaacaguauucuugaauguuauaccccagcccaaaccauuucaacugaguuugcuguuaaauugaaaauugacuuagccaaccgagagacaagcaucuucaguuaccgugaagaucccauugucuaugaaauucauccaaccaaaucuuuuauuaggcaugucaacaucgcucuaauucagagauaaucuguuguaccacuccuucccugcaacagcugaaucugcaacucccccugaaaaccaaagccuuuuucauguuagaugggauccuuuccaaauacuuugaucucauuuauguacauaauccuguguuuaagccuuuugaaaagccagugaugaucucaaugggcaaugaaaauguacuggaaauuaag

SEQ ID NO: 9 - RNA, 6537 nt, Artificial Sequence
  This sequence is synthesized in lab.
gcaggugacccggaggcccucgccgcccgcggcgccccgagcgcuuugugagcagaugcggagccgaguggagggcgcgagccagaugcggggcgacagcugacuugcugagaggaggcggggaggcgcggagcgcgcgugugguccuugcgccgcugacuucuccacugguuccugggcaccgaaagauaaaccucucauaaugaaggcccccgcugugcuugcaccuggcauccucgugcuccuguuuaccuuggugcagaggagcaauggggaguguaaagaggcacuagcaaaguccgagaugaaugugaauaugaaguaucagcuucccaacuucaccgcggaaacacccauccagaaugucauucuacaugagcaucacauuuuccuuggugccacuaacuacauuuauguuuuaaaugaggaagaccuucagaagguugcugaguacaagacugggccugugcuggaacacccagauuguuucccaugucaggacugcagcagcaaagccaauuuaucaggagguguuuggaaagauaacaucaacauggcucuaguugucgacaccuacuaugaugaucaacucauuagcuguggcagcgucaacagagggaccugccagcgacaugucuuuccccacaaucauacugcugacauacagucggagguucacugcauauucuccccacagauagaagagcccagccaguguccugacuguguggugagcgcccugggagccaaaguccuuucaucuguaaaggaccgguucaucaacuucuuuguaggcaauaccauaaauucuucuuauuucccagaucauccauugcauucgauaucagugagaaggcuaaaggaaacgaaagaugguuuuauguuuuugacggaccaguccuacauugauguuuuaccugaguucagagauucuuaccccauuaaguauguccaugccuuugaaagcaacaauuuuauuuacuucuugacgguccaaagggaaacucuagaugcucagacuuuucacacaagaauaaucagguucuguuccauaaacucuggauugcauuccuacauggaaaugccucuggaguguauucucacagaaaagagaaaaaagagauccacaaagaaggaaguguuuaauauacuucaggcugcguaugucagcaagccuggggcccagcuugcuagacaaauaggagccagccugaaugaugacauucuuuucgggguguucgcacaaagcaagccagauucugccgaaccaauggaucgaucugccaugugugcauucccuaucaaauaugucaacgacuucuucaacaagaucgucaacaaaaacaaugugagaugucuccagcauuuuuacggacccaaucaugagcacugcuuuaauaggacacuucugagaaauucaucaggcugugaagcgcgccgugaugaauaucgaacagaguuuaccacagcuuugcagcgcguugacuuauucaugggucaauucagcgaaguccucuuaacaucuauauccaccuucauuaaaggagaccucaccauagcuaaucuugggacaucagagggucgcuucaugcagguugugguuucucgaucaggaccaucaaccccucaugugaauuuucuccuggacucccauccagugucuccagaagugauuguggagcauacauuaaaccaaaauggcuacacacugguuaucacugggaagaagaucacgaagaucccauugaauggcuugggcugcagacauuuccaguccugcagucaaugccucucugccccacccuuuguucaguguggcuggugccacgacaaaugugugcgaucggaggaaugccugagcgggacauggacucaacagaucugucugccugcaaucuacaagguuuucccaaauagugcaccccuugaaggagggacaaggcugaccauauguggcugggacuuuggauuucggaggaauaauaaauuugauuuaaagaaaacuagaguucuccuuggaaaugagagcugcaccuugacuuuaagugagagcacgaugaauacauugaaaugcacaguugguccugccaugaauaagcauuucaauauguccauaauuauuucaaauggccacgggacaacacaauacaguacauucuccuauguggauccuguaauaacaaguauuucgccgaaauacgguccuauggcugguggcacuuuacuuacuuuaacuggaaauuaccuaaacagugggaauucuagacacauuucaauugguggaaaaacauguacuuuaaaaagugugucaaacaguauucuugaauguuauaccccagcccaaaccauuucaacugaguuugcuguuaaauugaaaauugacuuagccaaccgagagacaagcaucuucaguuaccgugaagaucccauugucuaugaaauucauccaaccaaaucuuuuauuaggcaugucaacaucgcucuaauucagagauaaucuguuguaccacuccuucccugcaacagcugaaucugcaacucccccugaaaaccaaagccuuuuucauguuagaugggauccuuuccaaauacuuugaucucauuuauguacauaauccuguguuuaagccuuuugaaaagccagugaugaucucaaugggcaaugaaaauguacuggaaauuaagggaaaugauauugacccugaagcaguuaaaggugaaguguuaaaaguuggaaauaagagcugugagaauauacacuuacauucugaagccguuuuaugcacgguccccaaugaccugcugaaauugaacagcgagcuaaauauagaguggaagcaagcaauuucuucaaccguccuuggaaaaguaauaguucaaccagaucagaauuucacaggauugauugcugguguugucucaauaucaacagcacuguuauuacuacuuggguuuuuccuguggcugaaaaagagaaagcaaauuaaagaucugggcagugaauuaguucgcuacgaugcaagaguacacacuccucauuuggauaggcuuguaagugcccgaaguguaagcccaacuacagaaaugguuucaaaugaaucuguagacuaccgagcuacuuuuccagaagaucaguuuccuaauucaucucagaacgguucaugccgacaagugcaguauccucugacagacaugucccccauccuaacuaguggggacucugauauauccaguccauuacugcaaaauacuguccacauugaccucagugcucuaaauccagagcugguccaggcagugcagcauguagugauugggcccaguagccugauugugcauuucaaugaagucauaggaagagggcauuuugguuguguauaucaugggacuuuguuggacaaugauggcaagaaaauucacugugcugugaaauccuugaacagaaucacugacauaggagaaguuucccaauuucugaccgagggaaucaucaugaaagauuuuagucaucccaauguccucucgcuccugggaaucugccugcgaagugaagggucuccgcuggugguccuaccauacaugaaacauggagaucuucgaaauuucauucgaaaugagacucauaauccaacuguaaaagaucuuauuggcuuuggucuucaaguagccaaaggcaugaaauaucuugcaagcaaaaaguuuguccacagagacuuggcugcaagaaacuguaugcuggaugaaaaauucacagucaagguugcugauuuuggucuugccagagacauguaugauaaagaauacuauaguguacacaacaaaacaggugcaaagcugccagugaaguggauggcuuuggaaagucugcaaacucaaaaguuuaccaccaagucagaugugugguccuuuggcgugcuccucugggagcugaugacaagaggagccccaccuuauccugacguaaacaccuuugauauaacuguuuacuuguugcaagggagaagacuccuacaacccgaauacugcccagaccccuuauaugaaguaaugcuaaaaugcuggcacccuaaagccgaaaugcgcccauccuuuucugaacuggugucccggauaucagcgaucuucucuacuuucauuggggagcacuauguccaugugaacgcuacuuaugugaacguaaaaugugucgcuccguauccuucucuguugucaucagaagauaacgcugaugaugagguggacacacgaccagccuccuucugggagacaucauagugcuaguacuaugucaaagcaacaguccacacuuuguccaaugguuuuuucacugccugaccuuuaaaaggccaucgauauucuuugcucuugccaaaauugcacuauuauaggacuuguauuguuauuuaaauuacuggauucuaaggaauuucuuaucugacagagcaucagaaccagaggcuuggucccacaggccacggaccaauggccugcagccgugacaacacuccugucauauuggaguccaaaacuugaauucuggguugaauuuuuuaaaaaucagguaccacuugauuucauaugggaaauugaagcaggaaauauugagggcuucuugaucacagaaaacucagaagagauaguaaugcucaggacaggagcggcagccccagaacaggccacucauuuagaauucuaguguuucaaaacacuuuuguguguuguauggucaauaacauuuuucauuacugauggugucauucacccauuagguaaacauucccuuuuaaauguuuguuuguuuuuugagacaggaucucacucuguugccagggcuguagugcaguggugugaucauagcucacugcaaccuccaccucccaggcucaagccucccgaauagcugggacuacaggcgcacaccaccauccccggcuaauuuuuguauuuuuuguagagacgggguuuugccauguugccaaggcugguuucaaacuccuggacucaagaaauccacccaccucagccucccaaagugcuaggauuacaggcaugagccacugcgcccagcccuuauaaauuuuuguauagacauuccuuugguuggaagaauauuuauaggcaauacagucaaaguuucaaaauagcaucacacaaaacauguuuauaaaugaacaggauguaauguacauagaugacauuaagaaaauuuguaugaaauaauuuagucaucaugaaauauuuaguugucauauaaaaacccacuguuugagaaugaugcuacucugaucuaaugaaugugaacauguagauguuuuguguguauuuuuuuaaaugaaaacucaaaauaagacaaguaauuuguugauaaauauuuuuaaagauaacucagcauguuuguaaagcaggauacauuuuacuaaaagguucauugguuccaaucacagcucauagguagagcaaagaaaggguggauggauugaaaagauuagccucugucucgguggcagguucccaccucgcaagcaauuggaaacaaaacuuuuggggaguuuuauuuugcauuaggguguguuuuauguuaagcaaaacauacuuuagaaacaaaugaaaaaggcaauugaaaaucccagcuauuucaccuagauggaauagccacccugagcagaacuuugugaugcuucauucuguggaauuuugugcuugcuacuguauagugcaugugguguagguuacucuaacugguuuugucgacguaaacauuuaaaguguuauauuuuuuauaaaaauguuuauuuuuaaugauaugagaaaaauuuuguuaggccacaaaaacacugcacugugaacauuuuagaaaagguaugucagacugggauuaaugacagcaugauuuucaaugacuguaaauugcgauaaggaaauguacugauugccaauacaccccacccucauuacaucaucaggacuugaagccaaggguuaacccagcaagcuacaaagagggugugucacacugaaacucaauaguugaguuuggcuguuguugcaggaaaaugauuauaacuaaaagcucucugauagugcagagacuuaccagaagacacaaggaauuguacugaagagcuauuacaauccaaauauugccguuucauaaauguaauaaguaauacuaauucacagaguauuguaaaugguggaugacaaaagaaaaucugcucuguggaaagaaagaacugucucuaccagggucaagagcaugaacgcaucaauagaaagaacucggggaaacaucccaucaacaggacuacacacuuguauauacauucuugagaacacugcaaugugaaaaucacguuugcuauuuauaaacuuguccuuagauuaaugugucuggacagauugugggaguaagugauucuucuaagaauuagauacuugucacugccuauaccugcagcugaacugaaugguacuucguauguuaauaguuguucugauaaaucaugcaauuaaaguaaagugaugcaacaucuugua

SEQ ID NO: 10 - RNA, 60 nt, Artificial Sequence
  This sequence is synthesized in lab.
cacagagacuuggcugcaagaaacuguauugguccuuuggcgugcuccucugggagcuga

SEQ ID NO: 11 - RNA, 247 nt, Artificial Sequence
  This sequence is synthesized in lab.
aauccaacuguaaaagaucuuauuggcuuuggucuucaaguagccaaaggcaugaaauaucuugcaagcaaaaaguuuguccacagagacuuggcugcaagaaacuguauugguccuuuggcgugcuccucugggagcugaugacaagaggagccccaccuuauccugacguaaacaccuuugauauaacuguuuacuuguugcaagggagaagacuccuacaacccgaauacugcccagaccccuu

SEQ ID NO: 12 - RNA, 6471 nt, Artificial Sequence
  This sequence is synthesized in lab.
gcaggugacccggaggcccucgccgcccgcggcgccccgagcgcuuugugagcagaugcggagccgaguggagggcgcgagccagaugcggggcgacagcugacuugcugagaggaggcggggaggcgcggagcgcgcgugugguccuugcgccgcugacuucuccacugguuccugggcaccgaaagauaaaccucucauaaugaaggcccccgcugugcuugcaccuggcauccucgugcuccuguuuaccuuggugcagaggagcaauggggaguguaaagaggcacuagcaaaguccgagaugaaugugaauaugaaguaucagcuucccaacuucaccgcggaaacacccauccagaaugucauucuacaugagcaucacauuuuccuuggugccacuaacuacauuuauguuuuaaaugaggaagaccuucagaagguugcugaguacaagacugggccugugcuggaacacccagauuguuucccaugucaggacugcagcagcaaagccaauuuaucaggagguguuuggaaagauaacaucaacauggcucuaguugucgacaccuacuaugaugaucaacucauuagcuguggcagcgucaacagagggaccugccagcgacaugucuuuccccacaaucauacugcugacauacagucggagguucacugcauauucuccccacagauagaagagcccagccaguguccugacuguguggugagcgcccugggagccaaaguccuuucaucuguaaaggaccgguucaucaacuucuuuguaggcaauaccauaaauucuucuuauuucccagaucauccauugcauucgauaucagugagaaggcuaaaggaaacgaaagaugguuuuauguuuuugacggaccaguccuacauugauguuuuaccugaguucagagauucuuaccccauuaaguauguccaugccuuugaaagcaacaauuuuauuuacuucuugacgguccaaagggaaacucuagaugcucagacuuuucacacaagaauaaucagguucuguuccauaaacucuggauugcauuccuacauggaaaugccucuggaguguauucucacagaaaagagaaaaaagagauccacaaagaaggaaguguuuaauauacuucaggcugcguaugucagcaagccuggggcccagcuugcuagacaaauaggagccagccugaaugaugacauucuuuucgggguguucgcacaaagcaagccagauucugccgaaccaauggaucgaucugccaugugugcauucccuaucaaauaugucaacgacuucuucaacaagaucgucaacaaaaacaaugugagaugucuccagcauuuuuacggacccaaucaugagcacugcuuuaauaggacacuucugagaaauucaucaggcugugaagcgcgccgugaugaauaucgaacagaguuuaccacagcuuugcagcgcguugacuuauucaugggucaauucagcgaaguccucuuaacaucuauauccaccuucauuaaaggagaccucaccauagcuaaucuugggacaucagagggucgcuucaugcagguugugguuucucgaucaggaccaucaaccccucaugugaauuuucuccuggacucccauccagugucuccagaagugauuguggagcauacauuaaaccaaaauggcuacacacugguuaucacugggaagaagaucacgaagaucccauugaauggcuugggcugcagacauuuccaguccugcagucaaugccucucugccccacccuuuguucaguguggcuggugccacgacaaaugugugcgaucggaggaaugccugagcgggacauggacucaacagaucugucugccugcaaucuacaagguuuucccaaauagugcaccccuugaaggagggacaaggcugaccauauguggcugggacuuuggauuucggaggaauaauaaauuugauuuaaagaaaacuagaguucuccuuggaaaugagagcugcaccuugacuuuaagugagagcacgaugaauacauugaaaugcacaguugguccugccaugaauaagcauuucaauauguccauaauuauuucaaauggccacgggacaacacaauacaguacauucuccuauguggauccuguaauaacaaguauuucgccgaaauacgguccuauggcugguggcacuuuacuuacuuuaacuggaaauuaccuaaacagugggaauucuagacacauuucaauugguggaaaaacauguacuuuaaaaagugugucaaacaguauucuugaauguuauaccccagcccaaaccauuucaacugaguuugcuguuaaauugaaaauugacuuagccaaccgagagacaagcaucuucaguuaccgugaagaucccauugucuaugaaauucauccaaccaaaucuuuuauuaguggugggagcacaauaacagguguugggaaaaaccugaauucaguuagugucccgagaauggucauaaaugugcaugaagcaggaaggaacuuuacaguggcaugucaacaucgcucuaauucagagauaaucuguuguaccacuccuucccugcaacagcugaaucugcaacucccccugaaaaccaaagccuuuuucauguuagaugggauccuuuccaaauacuuugaucucauuuauguacauaauccuguguuuaagccuuuugaaaagccagugaugaucucaaugggcaaugaaaauguacuggaaauuaagggaaaugauauugacccugaagcaguuaaaggugaaguguuaaaaguuggaaauaagagcugugagaauauacacuuacauucugaagccguuuuaugcacgguccccaaugaccugcugaaauugaacagcgagcuaaauauagaguggaagcaagcaauuucuucaaccguccuuggaaaaguaauaguucaaccagaucagaauuucacaggauugauugcugguguugucucaauaucaacagcacuguuauuacuacuuggguuuuuccuguggcugaaaaagagaaagcaaauuaaagaucugggcagugaauuaguucgcuacgaugcaagaguacacacuccucauuuggauaggcuuguaagugcccgaaguguaagcccaacuacagaaaugguuucaaaugaaucuguagacuaccgagcuacuuuuccagaagaucaguuuccuaauucaucucagaacgguucaugccgacaagugcaguauccucugacagacaugucccccauccuaacuaguggggacucugauauauccaguccauuacugcaaaauacuguccacauugaccucagugcucuaaauccagagcugguccaggcagugcagcauguagugauugggcccaguagccugauugugcauuucaaugaagucauaggaagagggcauuuugguuguguauaucaugggacuuuguuggacaaugauggcaagaaaauucacugugcugugaaauccuugaacagaaucacugacauaggagaaguuucccaauuucugaccgagggaaucaucaugaaagauuuuagucaucccaauguccucucgcuccugggaaucugccugcgaagugaagggucuccgcuggugguccuaccauacaugaaacauggagaucuucgaaauuucauucgaaaugagacucauaauccaacuguaaaagaucuuauuggcuuuggucuucaaguagccaaaggcaugaaauaucuugcaagcaaaaaguuuguccacagagacuuggcugcaagaaacuguauugguccuuuggcgugcuccucugggagcugaugacaagaggagccccaccuuauccugacguaaacaccuuugauauaacuguuuacuuguugcaagggagaagacuccuacaacccgaauacugcccagaccccuuauaugaaguaaugcuaaaaugcuggcacccuaaagccgaaaugcgcccauccuuuucugaacuggugucccggauaucagcgaucuucucuacuuucauuggggagcacuauguccaugugaacgcuacuuaugugaacguaaaaugugucgcuccguauccuucucuguugucaucagaagauaacgcugaugaugagguggacacacgaccagccuccuucugggagacaucauagugcuaguacuaugucaaagcaacaguccacacuuuguccaaugguuuuuucacugccugaccuuuaaaaggccaucgauauucuuugcucuugccaaaauugcacuauuauaggacuuguauuguuauuuaaauuacuggauucuaaggaauuucuuaucugacagagcaucagaaccagaggcuuggucccacaggccacggaccaauggccugcagccgugacaacacuccugucauauuggaguccaaaacuugaauucuggguugaauuuuuuaaaaaucagguaccacuugauuucauaugggaaauugaagcaggaaauauugagggcuucuugaucacagaaaacucagaagagauaguaaugcucaggacaggagcggcagccccagaacaggccacucauuuagaauucuaguguuucaaaacacuuuuguguguuguauggucaauaacauuuuucauuacugauggugucauucacccauuagguaaacauucccuuuuaaauguuuguuuguuuuuugagacaggaucucacucuguugccagggcuguagugcaguggugugaucauagcucacugcaaccuccaccucccaggcucaagccucccgaauagcugggacuacaggcgcacaccaccauccccggcuaauuuuuguauuuuuuguagagacgggguuuugccauguugccaaggcugguuucaaacuccuggacucaagaaauccacccaccucagccucccaaagugcuaggauuacaggcaugagccacugcgcccagcccuuauaaauuuuuguauagacauuccuuugguuggaagaauauuuauaggcaauacagucaaaguuucaaaauagcaucacacaaaacauguuuauaaaugaacaggauguaauguacauagaugacauuaagaaaauuuguaugaaauaauuuagucaucaugaaauauuuaguugucauauaaaaacccacuguuugagaaugaugcuacucugaucuaaugaaugugaacauguagauguuuuguguguauuuuuuuaaaugaaaacucaaaauaagacaaguaauuuguugauaaauauuuuuaaagauaacucagcauguuuguaaagcaggauacauuuuacuaaaagguucauugguuccaaucacagcucauagguagagcaaagaaaggguggauggauugaaaagauuagccucugucucgguggcagguucccaccucgcaagcaauuggaaacaaaacuuuuggggaguuuuauuuugcauuaggguguguuuuauguuaagcaaaacauacuuuagaaacaaaugaaaaaggcaauugaaaaucccagcuauuucaccuagauggaauagccacccugagcagaacuuugugaugcuucauucuguggaauuuugugcuugcuacuguauagugcaugugguguagguuacucuaacugguuuugucgacguaaacauuuaaaguguuauauuuuuuauaaaaauguuuauuuuuaaugauaugagaaaaauuuuguuaggccacaaaaacacugcacugugaacauuuuagaaaagguaugucagacugggauuaaugacagcaugauuuucaaugacuguaaauugcgauaaggaaauguacugauugccaauacaccccacccucauuacaucaucaggacuugaagccaaggguuaacccagcaagcuacaaagagggugugucacacugaaacucaauaguugaguuuggcuguuguugcaggaaaaugauuauaacuaaaagcucucugauagugcagagacuuaccagaagacacaaggaauuguacugaagagcuauuacaauccaaauauugccguuucauaaauguaauaaguaauacuaauucacagaguauuguaaaugguggaugacaaaagaaaaucugcucuguggaaagaaagaacugucucuaccagggucaagagcaugaacgcaucaauagaaagaacucggggaaacaucccaucaacaggacuacacacuuguauauacauucuugagaacacugcaaugugaaaaucacguuugcuauuuauaaacuuguccuuagauuaaugugucuggacagauugugggaguaagugauucuucuaagaauuagauacuugucacugccuauaccugcagcugaacugaaugguacuucguauguuaauaguuguucugauaaaucaugcaauuaaaguaaagugaugcaacaucuugua

SEQ ID NO: 13 - protein, 21 aa, Artificial Sequence
  This sequence is synthesized in lab.
VYEIHPTKSFIRHVNIALIQR

SEQ ID NO: 14 - protein, 51 aa, Artificial Sequence
  This sequence is synthesized in lab.
QTISTEFAVKLKIDLANRETSIFSYREDPIVYEIHPTKSFIRHVNIALIQR

SEQ ID NO: 15 - protein, 764 aa, Artificial Sequence
  This sequence is synthesized in lab.
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFIRHVNIALIQR

SEQ ID NO: 16 - protein, 20 aa, Artificial Sequence
  This sequence is synthesized in lab.
VHRDLAARNCIGPLACSSGS

SEQ ID NO: 17 - protein, 50 aa, Artificial Sequence
  This sequence is synthesized in lab.
NETHNPTVKDLIGFGLQVAKGMKYLASKKFVHRDLAARNCIGPLACSSGS

SEQ ID NO: 18 - protein, 1220 aa, Artificial Sequence
  This sequence is synthesized in lab.
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCIGPLACSSGS

SEQ ID NO: 19 - DNA, 27 bp, Artificial Sequence
  This sequence is synthesized in lab.
atccaaccaaatcttttattaggcatg

SEQ ID NO: 20 - DNA, 26 bp, Artificial Sequence
  This sequence is synthesized in lab.
cttctggaaaagtagctcggtagtct

SEQ ID NO: 21 - DNA, 28 bp, Artificial Sequence
  This sequence is synthesized in lab.
caaatcttttattaggcatgtcaacatc

SEQ ID NO: 22 - DNA, 22 bp, Artificial Sequence
  This sequence is synthesized in lab.
aatttctgaccgagggaatcat

SEQ ID NO: 23 - DNA, 24 bp, Artificial Sequence
  This sequence is synthesized in lab.
acgccaaaggaccaatacagtttc

SEQ ID NO: 24 - DNA, 24 bp, Artificial Sequence
  This sequence is synthesized in lab.
gccaaaggaccaatacagtttctt

SEQ ID NO: 25 - DNA, 47 bp, Artificial Sequence
  This sequence is synthesized in lab.
taatacgactcactatagggagcgatgttgacatgcctaataaaaga

SEQ ID NO: 26 - DNA, 21 bp, Artificial Sequence
  This sequence is synthesized in lab.
gtgtgtcaaacagtattcttg

SEQ ID NO: 27 - DNA, 28 bp, Artificial Sequence
  This sequence is synthesized in lab.
ttgacatgcctaataaaagatttggttg

SEQ ID NO: 28 - DNA, 43 bp, Artificial Sequence
  This sequence is synthesized in lab.
taatacgactcactataggggagcacgccaaaggaccaataca

SEQ ID NO: 29 - DNA, 25 bp, Artificial Sequence
  This sequence is synthesized in lab.
tgaaatatcttgcaagcaaaaagtt

SEQ ID NO: 30 - DNA, 24 bp, Artificial Sequence
  This sequence is synthesized in lab.
agcacgccaaaggaccaatacagt

(19) United States
(12) Patent Application Publication
(10) Pub. No.: US 2023/0160007 A1
(43) Pub. Date: May 25, 2023

(54) KIT FOR EVALUATING SENSITIVITY OF PATIENT TO MET INHIBITOR

(71) Applicant: Beijing Neurosurgical Institute, Beijing (CN)

(72) Inventors: Tao JIANG, Beijing (CN); Ruichao CHAI, Beijing (CN); Zheng ZHAO, Beijing (CN); Jing CHEN, Beijing (CN); Kenan ZHANG, Beijing (CN)

(73) Assignee: Beijing Neurosurgical Institute, Beijing (CN)

(21) Appl. No.: 17/919,704

(22) PCT Filed: Aug. 24, 2021
(86) PCT No.: PCT/CN2021/114400

§ 371 Date: Oct. 18, 2022

(30) Foreign Application Priority Data
Aug. 26, 2020 (CN) 202010872686.9

(51) Int. Cl. C12Q 1/6876; C12Q 1/6844

(52) U.S. Cl. CPC C12Q 1/6876; C12Q 1/6844; C12Q 2600/106

(57) ABSTRACT

A kit for evaluating the sensitivity of a patient to an MET inhibitor is provided. The kit includes a first primer pair capable of specifically amplifying a first nucleotide sequence, and/or a first probe capable of being specifically bonded to the first nucleotide sequence, wherein the first nucleotide sequence is as shown in SEQ ID NO: 1, and/or the kit comprises a second primer pair capable of specifically amplifying a second nucleotide sequence, and/or a second probe capable of being specifically bonded to the second nucleotide sequence, wherein the second nucleotide sequence is as shown in SEQ ID NO: 4. The kit provided is capable of screening the patients sensitive to an MET inhibitor.

### TECHNICAL FIELD

The disclosure relates to the field of biotechnology, in particular to a kit for evaluating the sensitivity of patient to MET inhibitor.

### BACKGROUND

The mesenchymal epithelial transition factor (MET) gene, located on the long arm of human chromosome 7, contains 21 exons and is a receptor tyrosine kinase for hepatocyte growth factor (HGF), and RAS-MAPK and PI3K-AKT signaling pathways participating in tumor generation and metastasis can be activated by combining the MET with the HGF. C-MET is a transmembrane receptor which is encoded by the MET gene and has an autophosphorylation activity, and is a key factor for embryonic development, organ regeneration and wound healing. Abnormal forms of the MET gene include mutation, amplification, rearrangement and overexpression, and MET gene abnormality is an important factor causing various tumors.

MET inhibitors can effectively treat various tumors caused by MET gene abnormality. However, different patients have different sensitivities to MET inhibitors, so that a treatment effect varies from person to person, and thus, patients with a better sensitivity to MET inhibitors need to be screened for treatment to achieve a good treatment effect.

### SUMMARY

The disclosure aims to provide a kit for evaluating the sensitivity of a patient to an MET inhibitor, and the kit can be used for screening patients with better sensitivity to the MET inhibitor, so that the patients can be effectively treated.

In order to achieve the purpose, in a first aspect, the disclosure provides a kit for evaluating the sensitivity of a patient to an MET inhibitor, where the kit includes a first primer pair capable of specifically amplifying a first nucleotide sequence, and/or a first probe capable of specifically binding to the first nucleotide sequence, and the first nucleotide sequence is as shown in SEQ ID NO. 1; and/or,

the kit includes a second primer pair capable of specifically amplifying a second nucleotide sequence, and/or a second probe capable of specifically binding to the second nucleotide sequence, and the second nucleotide sequence is shown as SEQ ID NO. 4.

Optionally, the first nucleotide sequence is as shown in SEQ ID NO. 2; preferably, the first nucleotide sequence is as shown in SEQ ID NO. 3, where the nucleotide sequence as shown in SEQ ID NO. 3 is a cDNA sequence of the MET gene with an exon 10 deleted.

The second nucleotide sequence is as shown in SEQ ID NO. 5; preferably, the second nucleotide sequence is as shown in SEQ ID NO. 6, where the nucleotide sequence as shown in SEQ ID NO. 6 is a cDNA sequence of the MET gene with an exon 19 deleted.

Optionally, the patient includes a glioma patient and/or a gastric adenocarcinoma patient.

In a second aspect, the disclosure provides a kit for evaluating the sensitivity of a patient to an MET inhibitor, where the kit includes a third primer pair capable of specifically amplifying a third nucleotide sequence, and/or a third probe capable of specifically binding to the third nucleotide sequence, and the third nucleotide sequence is as shown in SEQ ID NO. 7; and/or,

the kit includes a fourth primer pair capable of specifically amplifying a fourth nucleotide sequence, and/or a fourth probe capable of specifically binding to the fourth nucleotide sequence, and the fourth nucleotide sequence is shown as SEQ ID NO. 10.

Optionally, the third nucleotide sequence is as shown in SEQ ID NO. 8; preferably, the third nucleotide sequence is as shown in SEQ ID NO. 9, where the nucleotide sequence as shown in SEQ ID NO. 9 is an mRNA sequence of the MET gene with an exon 10 deleted.

The fourth nucleotide sequence is as shown in SEQ ID NO. 11; preferably, the fourth nucleotide sequence is as shown in SEQ ID NO. 12, where the nucleotide sequence as shown in SEQ ID NO. 12 is an mRNA sequence of the MET gene with an exon 19 deleted.

In a third aspect, the disclosure provides a kit for evaluating the sensitivity of a patient to an MET inhibitor, where the kit includes a first antibody for resisting a first amino acid sequence, and the first amino acid sequence is shown as SEQ ID NO. 13; and/or,

the kit includes a second antibody for resisting a second amino acid sequence, and the second amino acid sequence is shown as SEQ ID NO. 16.

Optionally, the first amino acid sequence is as shown in SEQ ID NO. 14; preferably, the first amino acid sequence is as shown in SEQ ID NO. 15, where the amino acid sequence as shown in SEQ ID NO. 15 is encoded by mRNA as shown in SEQ ID NO. 9.

The second amino acid sequence is as shown in SEQ ID NO. 17; preferably, the second amino acid sequence is as shown in SEQ ID NO. 18, where the amino acid sequence as shown in SEQ ID NO. 18 is encoded by mRNA as shown in SEQ ID NO. 12.

In a fourth aspect, the disclosure provides application of a molecular reagent in preparation of a kit for evaluating the sensitivity of a patient to an MET inhibitor, where the molecular reagent includes at least one of (1)-(10) as follows:

(1) a first primer pair capable of specifically amplifying a first nucleotide sequence;

(2) a first probe capable of specifically binding to the first nucleotide sequence;

where the first nucleotide sequence is as shown in SEQ ID NO. 1;

(3) a second primer pair capable of specifically amplifying a second nucleotide sequence;

(4) a second probe capable of specifically binding to the second nucleotide sequence;

where the second nucleotide sequence is as shown in SEQ ID NO. 4;

(5) a third primer pair capable of specifically amplifying a third nucleotide sequence;

(6) a third probe capable of specifically binding to the third nucleotide sequence;

where the third nucleotide sequence is as shown in SEQ ID NO. 7;

(7) a fourth primer pair capable of specifically amplifying a fourth nucleotide sequence;

(8) a fourth probe capable of specifically binding to the fourth nucleotide sequence;

where the fourth nucleotide sequence is as shown in SEQ ID NO. 10;

(9) a first antibody for resisting a first amino acid sequence;

where the first amino acid sequence is as shown in SEQ ID NO. 13; and

(10) a second antibody for resisting a second amino acid sequence;

where the second amino acid sequence is as shown in SEQ ID NO. 16.

Optionally, the first nucleotide sequence is as shown in SEQ ID NO. 2; preferably, the first nucleotide sequence is as shown in SEQ ID NO. 3, where the nucleotide sequence as shown in SEQ ID NO. 3 is a cDNA sequence of the MET gene with an exon 10 deleted.

The second nucleotide sequence is as shown in SEQ ID NO. 5; preferably, the second nucleotide sequence is as shown in SEQ ID NO. 6, where the nucleotide sequence as shown in SEQ ID NO. 6 is a cDNA sequence of the MET gene with an exon 19 deleted.

The third nucleotide sequence is as shown in SEQ ID NO. 8; preferably, the third nucleotide sequence is as shown in SEQ ID NO. 9, where the nucleotide sequence as shown in SEQ ID NO. 9 is an mRNA sequence of the MET gene with an exon 10 deleted.

The fourth nucleotide sequence is as shown in SEQ ID NO. 11; preferably, the fourth nucleotide sequence is as shown in SEQ ID NO. 12, where the nucleotide sequence as shown in SEQ ID NO. 12 is an mRNA sequence of the MET gene with the an 19 deleted.

The first amino acid sequence is as shown in SEQ ID NO. 14; preferably, the first amino acid sequence is as shown in SEQ ID NO. 15, where the amino acid sequence as shown in SEQ ID NO. 15 is encoded by mRNA as shown in SEQ ID NO. 9.

The second amino acid sequence is as shown in SEQ ID NO. 17; preferably, the second amino acid sequence is as shown in SEQ ID NO. 18, where the amino acid sequence as shown in SEQ ID NO. 18 is encoded by mRNA as shown in SEQ ID NO. 12.

In a fifth aspect, the disclosure provides a system for evaluating the sensitivity of a patient to an MET inhibitor, where the system includes an amplification device, a sequencing device, a computing device, and an output device.

The amplification device includes a collection unit and an amplification unit, where the collection unit is used for collecting a template nucleic acid fragment and an amplification primer, and the amplification unit is used for amplifying the template nucleic acid fragment by using the amplification primer to obtain an amplification product.

The sequencing device is used for sequencing the nucleic acid sequence of the amplification product to obtain a sequence of the amplification product.

The computing device includes a memory and a processor, where a computer program is stored in the memory, and the processor is configured to execute the computer program stored in the memory so as to achieve the following determination:

if the sequence of the amplification product contains at least one of a first nucleotide sequence, a second nucleotide sequence, a third nucleotide sequence or a fourth nucleotide sequence, it is determined that the patient is sensitive to the MET inhibitor.

Where the first nucleotide sequence is as shown in SEQ ID NO. 1; preferably, the first nucleotide sequence is as shown in SEQ ID NO. 2; more preferably, the first nucleotide sequence is as shown in SEQ ID NO. 3, and the nucleotide sequence as shown in SEQ ID NO. 3 is a cDNA sequence of the MET gene with an exon 10 deleted.

The second nucleotide sequence is as shown in SEQ ID NO. 4; preferably, the second nucleotide sequence is as shown in SEQ ID NO. 5; more preferably, the second nucleotide sequence is as shown in SEQ ID NO. 6, where the nucleotide sequence as shown in SEQ ID NO. 6 is a cDNA sequence of the MET gene with an exon 19 deleted.

The third nucleotide sequence is as shown in SEQ ID NO. 7; preferably, the third nucleotide sequence is as shown in SEQ ID NO. 8; more preferably, the third nucleotide sequence is as shown in SEQ ID NO. 9, where the nucleotide sequence as shown in SEQ ID NO. 9 is an mRNA sequence of the MET gene with an exon 10 deleted.

The fourth nucleotide sequence is as shown in SEQ ID NO. 10; preferably, the fourth nucleotide sequence is as shown in SEQ ID NO. 11; more preferably, the fourth nucleotide sequence is as shown in SEQ ID NO. 12, where the nucleotide sequence as shown in SEQ ID NO. 12 is an mRNA sequence of the MET gene with an exon 19 deleted.

In a sixth aspect, the disclosure provides a method for evaluating the sensitivity of a patient to an MET inhibitor, where the method includes a step of detecting whether a tumor sample cell contains the first nucleotide sequence, the second nucleotide sequence, the third nucleotide sequence, the fourth nucleotide sequence, the first amino acid sequence or the second amino acid sequence, and if it is detected that the tumor sample cell contains at least one of the first nucleotide sequence, the second nucleotide sequence, the third nucleotide sequence, the fourth nucleotide sequence, the first amino acid sequence or the second amino acid sequence, it is determined that the patient corresponding to the tumor sample cell is sensitive to the MET inhibitor.

Through the technical solution, by utilizing the kit provided by the disclosure, patients sensitive to the MET inhibitor can be quickly screened out, so that the patients can be effectively treated.

Other features and advantages of the disclosure will be described in detail in the subsequent specific examples.

### BRIEF DESCRIPTION OF THE DRAWINGS

The drawings are used to provide a further understanding of the disclosure and constitute a part of the description, and are used to explain the disclosure together with the following specific examples, but do not constitute limitations of the disclosure. In the drawings:

FIG. 1 is a schematic diagram of the number of reads of exons 9-11 of the MET gene of a glioblastoma patient derived cell (PDC) sample A provided by the examples of the disclosure.

FIG. 2 is a schematic diagram of the number of reads of exons 9-11 of the MET gene of a glioblastoma PDC sample B provided by the examples of the disclosure.

FIG. 3 is a schematic diagram of the number of reads of exons 9-11 of the MET gene of a glioblastoma PDC sample C provided by the examples of the disclosure.

FIG. 4 is a schematic diagram of the number of reads of exons 9-11 of the MET gene of a glioblastoma PDC sample D provided by the examples of the disclosure;

FIG. 5 is a schematic diagram of the number of reads of exons 18-20 of the MET gene of a glioblastoma PDC sample A provided by the examples of the disclosure.

FIG. 6 is a schematic diagram of the number of reads of exons 18-20 of the MET gene of a glioblastoma PDC sample B provided by the examples of the disclosure.

FIG. 7 is a schematic diagram of the number of reads of exons 18-20 of the MET gene of a glioblastoma PDC sample C provided by the examples of the disclosure.

FIG. 8 is a schematic diagram of the number of reads of exons 18-20 of the MET gene of a glioblastoma PDC sample D provided by the examples of the disclosure.

FIG. 9 is an agarose gel electrophoretogram of PCR amplification products of glioblastoma PDC samples A, B, C and D provided by the examples of the disclosure.

FIG. 10 is an agarose gel electrophoretogram of PCR amplification products of another glioblastoma PDC samples A, B, C and D provided by the examples of the disclosure.

FIG. 11 is a curve graph showing relative cell survival of glioblastoma PDC samples treated by temozolomide (TMZ) with different concentrations provided by the examples of the disclosure.

FIG. 12 is a curve graph showing relative cell survival of glioblastoma PDC samples treated by crizotinib with different concentrations provided by the examples of the disclosure.

### DETAILED DESCRIPTION

The specific examples of the disclosure are described in detail below in combination with the drawings. It should be understood that the specific examples described herein are only used to illustrate and interpret the disclosure, but not to limit the disclosure.

In a first aspect, the disclosure provides a kit for evaluating the sensitivity of a patient to an MET inhibitor, where the kit includes a first primer pair capable of specifically amplifying a first nucleotide sequence, and/or a first probe capable of specifically binding to the first nucleotide sequence, and the first nucleotide sequence is as shown in SEQ ID NO. 1; and/or the kit includes a second primer pair capable of specifically amplifying a second nucleotide sequence, and/or a second probe capable of specifically binding to the second nucleotide sequence, and the second nucleotide sequence is as shown in SEQ ID NO. 4.

Optionally, the first nucleotide sequence is as shown in SEQ ID NO. 2; preferably, the first nucleotide sequence is as shown in SEQ ID NO. 3, where the nucleotide sequence as shown in SEQ ID NO. 3 is a cDNA sequence of the MET gene with an exon 10 deleted; the second nucleotide sequence is as shown in SEQ ID NO. 5; preferably, the second nucleotide sequence is as shown in SEQ ID NO. 6, where the nucleotide sequence as shown in SEQ ID NO. 6 is a cDNA sequence of the MET gene with an exon 19 deleted.

Optionally, the patient includes a glioma patient and/or a gastric adenocarcinoma patient.

In the above technical solution, the sequence as shown in SEQ ID NO. 1 is a cDNA fragment containing a deletion site of the exon 10 of the human MET gene. The sequence as shown in SEQ ID NO. 2 is a cDNA fragment containing the sequence as shown in SEQ ID NO. 1. The sequence as shown in SEQ ID NO. 4 is a cDNA fragment containing a deletion site of the exon 19 of the human MET gene. And the sequence as shown in SEQ ID NO. 5 is a cDNA fragment containing the sequence as shown in SEQ ID NO. 4.

The inventors of the disclosure found that tumor cells are more sensitive to the MET inhibitor when mRNA of tumor cells can reversely transcribe cDNA of the MET gene with the deletion site of the exon 10 and/or the exon 19, so that the sensitivity of the tumor cells to the MET inhibitor can be determined by detecting the deletion state of the exon 10 and the exon 19 of the cDNA of the MET gene in the tumor cells, and patients suitable for use of the MET inhibitor are screened out, so as to obtain the disclosure.

Where a first primer pair capable of specifically amplifying the nucleotide sequence as shown in SEQ ID NO. 1 can be used in the disclosure. For example, the first primer pair may contain an upstream primer as shown in SEQ ID NO. 19 and a downstream primer as shown in SEQ ID NO. 20. A first probe capable of specifically binding to the nucleotide sequence as shown in SEQ ID NO. 1 can be used in the disclosure. For example, a sequence of the first probe may contain a sequence as shown in SEQ ID NO. 21.

A second primer pair capable of specifically amplifying the nucleotide sequence as shown in SEQ ID NO. 4 can be used in the disclosure. For example, the second primer pair may contain an upstream primer as shown in SEQ ID NO. 22 and a downstream primer as shown in SEQ ID NO. 23. A second probe capable of specifically binding to the nucleotide sequence shown in SEQ ID NO. 4 can be used in the disclosure. For example, a sequence of the second probe may contain a sequence as shown in SEQ ID NO. 24.

In a second aspect, the disclosure provides a kit for evaluating the sensitivity of a patient to an MET inhibitor, where the kit includes a third primer pair capable of specifically amplifying a third nucleotide sequence, and/or a third probe capable of specifically binding to the third nucleotide sequence, and the third nucleotide sequence is as shown in SEQ ID NO. 7; and/or, the kit includes a fourth primer pair capable of specifically amplifying a fourth nucleotide sequence, and/or a fourth probe capable of specifically binding to the fourth nucleotide sequence, and the fourth nucleotide sequence is as shown in SEQ ID NO. 10.

Optionally, the third nucleotide sequence is as shown in SEQ ID NO. 8; preferably, the third nucleotide sequence is as shown in SEQ ID NO. 9, where the nucleotide sequence as shown in SEQ ID NO. 9 is an mRNA sequence of the MET gene with an exon 10 deleted; the fourth nucleotide sequence is as shown in SEQ ID NO. 11; preferably, the fourth nucleotide sequence is as shown in SEQ ID NO. 12, where the nucleotide sequence as shown in SEQ ID NO. 12 is an mRNA sequence of the MET gene with an exon 19 deleted.

In the above technical solution, the sequence as shown in SEQ ID NO. 7 is an mRNA fragment containing a deletion site of the exon 10 of the human MET gene. The sequence as shown in SEQ ID NO. 8 is an mRNA fragment containing the sequence as shown in SEQ ID NO. 7. The sequence as shown in SEQ ID NO. 10 is an mRNA fragment containing a deletion site of the exon 19 of the human MET gene. And the sequence as shown in SEQ ID NO. 11 is an mRNA fragment containing the sequence as shown in SEQ ID NO. 10.

Where a third primer pair capable of specifically amplifying the nucleotide sequence as shown in SEQ ID NO. 7 can be used in the disclosure. For example, the third primer pair may contain an upstream primer as shown in SEQ ID NO. 25 and a downstream primer as shown in SEQ ID NO. 26. A third probe capable of specifically binding to the nucleotide sequence shown in SEQ ID NO. 7 can be used in the disclosure. For example, a sequence of the third probe may contain a sequence shown in SEQ ID NO. 27.

A fourth primer pair capable of specifically amplifying the nucleotide sequence as shown in SEQ ID NO. 10 can be used in the disclosure. For example, the fourth primer pair may contain an upstream primer as shown in SEQ ID NO. 28 and a downstream primer as shown in SEQ ID NO. 29. A fourth probe capable of specifically binding to the nucleotide sequence shown in SEQ ID NO. 10 can be used in the disclosure. For example, a sequence of the fourth probe may contain a sequence shown in SEQ ID NO. 30.

In a third aspect, the disclosure provides a kit for evaluating the sensitivity of a patient to an MET inhibitor, where the kit includes a first antibody for resisting a first amino acid sequence, and the first amino acid sequence is shown as SEQ ID NO. 13; and/or the kit includes a second antibody for resisting a second amino acid sequence, and the second amino acid sequence is as shown in SEQ ID NO. 16.

Optionally, the first amino acid sequence is as shown in SEQ ID NO. 14; preferably, the first amino acid sequence is as shown in SEQ ID NO. 15, where the amino acid sequence as shown in SEQ ID NO. 15 is encoded by mRNA as shown in SEQ ID NO. 9; the second amino acid sequence is as shown in SEQ ID NO. 17; preferably, the second amino acid sequence is as shown in SEQ ID NO. 18, where the amino acid sequence as shown in SEQ ID NO. 18 is encoded by mRNA as shown in SEQ ID NO. 12.

Where the antibodies for resisting the first amino acid sequence and the second amino acid sequence can be a monoclonal antibody and/or a polyclonal antibody.

In a fourth aspect, the disclosure provides application of a molecular reagent in preparation of a kit for evaluating the sensitivity of a patient to an MET inhibitor. The molecular reagent includes at least one of (1)-(10) as follows: (1) a first primer pair capable of specifically amplifying a first nucleotide sequence; (2) a first probe capable of specifically binding to the first nucleotide sequence; where the first nucleotide sequence is as shown in SEQ ID NO. 1; (3) a second primer pair capable of specifically amplifying a second nucleotide sequence; (4) a second probe capable of specifically binding to the second nucleotide sequence; where the second nucleotide sequence is as shown in SEQ ID NO. 4; (5) a third primer pair capable of specifically amplifying a third nucleotide sequence; (6) a third probe capable of specifically binding to the third nucleotide sequence; where the third nucleotide sequence is as shown in SEQ ID NO. 7; (7) a fourth primer pair capable of specifically amplifying a fourth nucleotide sequence; (8) a fourth probe capable of specifically binding to the fourth nucleotide sequence; where the fourth nucleotide sequence is as shown in SEQ ID NO. 10; (9) a first antibody for resisting a first amino acid sequence; where the first amino acid sequence is as shown in SEQ ID NO. 13; and (10) a second antibody for resisting a second amino acid sequence; where the second amino acid sequence is as shown in SEQ ID NO. 16.

Optionally, the first nucleotide sequence is as shown in SEQ ID NO. 2; preferably, the first nucleotide sequence is as shown in SEQ ID NO. 3, where the nucleotide sequence as shown in SEQ ID NO. 3 is a cDNA sequence of the MET gene with an exon 10 deleted. The second nucleotide sequence is as shown in SEQ ID NO. 5; preferably, the second nucleotide sequence is as shown in SEQ ID NO. 6, where the nucleotide sequence as shown in SEQ ID NO. 6 is a cDNA sequence of the MET gene with an exon 19 deleted. The third nucleotide sequence is as shown in SEQ ID NO. 8; preferably, the third nucleotide sequence is as shown in SEQ ID NO. 9, where the nucleotide sequence as shown in SEQ ID NO. 9 is an mRNA sequence of the MET gene with an exon 10 deleted. The fourth nucleotide sequence is as shown in SEQ ID NO. 11; preferably, the fourth nucleotide sequence is as shown in SEQ ID NO. 12, where the nucleotide sequence as shown in SEQ ID NO. 12 is an mRNA sequence of the MET gene with an exon 19 deleted. The first amino acid sequence is as shown in SEQ ID NO. 14; preferably, the first amino acid sequence is as shown in SEQ ID NO. 15, where the amino acid sequence as shown in SEQ ID NO. 15 is encoded by mRNA as shown in SEQ ID NO. 9. The second amino acid sequence is as shown in SEQ ID NO. 17; preferably, the second amino acid sequence is as shown in SEQ ID NO. 18, where the amino acid sequence as shown in SEQ ID NO. 18 is encoded by mRNA as shown in SEQ ID NO. 12.

In a fifth aspect, the disclosure provides a system for evaluating the sensitivity of a patient to an MET inhibitor, where the system includes an amplification device, a sequencing device, a computing device, and an output device; the amplification device includes a collection unit and an amplification unit, where the collection unit is used for collecting a template nucleic acid fragment and an amplification primer, and the amplification unit is used for amplifying the template nucleic acid fragment by using the amplification primer to obtain an amplification product; the sequencing device is used for sequencing the nucleic acid sequence of the amplification product to obtain a sequence of the amplification product; the computing device includes a memory and a processor, where a computer program is stored in the memory and the processor is configured to execute the computer program stored in the memory, so as to achieve the following determination: if the sequence of the amplification product contains at least one of a first nucleotide sequence, a second nucleotide sequence, a third nucleotide sequence or a fourth nucleotide sequence, it is determined that the patient is sensitive to the MET inhibitor; where the first nucleotide sequence is as shown in SEQ ID NO. 1; preferably, the first nucleotide sequence is as shown in SEQ ID NO. 2; more preferably, the first nucleotide sequence is as shown in SEQ ID NO. 3, where the nucleotide sequence as shown in SEQ ID NO. 3 is a cDNA sequence of the MET gene with an exon 10 deleted; the second nucleotide sequence is as shown in SEQ ID NO. 4; preferably, the second nucleotide sequence is as shown in SEQ ID NO. 5; more preferably, the second nucleotide sequence is as shown in SEQ ID NO. 6, where the nucleotide sequence as shown in SEQ ID NO. 6 is a cDNA sequence of the MET gene with an exon 19 deleted; the third nucleotide sequence is as shown in SEQ ID NO. 7; preferably, the third nucleotide sequence is as shown in SEQ ID NO. 8; more preferably, the third nucleotide sequence is as shown in SEQ ID NO. 9, where the nucleotide sequence as shown in SEQ ID NO. 9 is an mRNA sequence of the MET gene with an exon 10 deleted; the fourth nucleotide sequence is as shown in SEQ ID NO. 10; preferably, the fourth nucleotide sequence is as shown in SEQ ID NO. 11; more preferably, the fourth nucleotide sequence is as shown in SEQ ID NO. 12, where the nucleotide sequence as shown in SEQ ID NO. 12 is an mRNA sequence of the MET gene with an exon 19 deleted.

In a sixth aspect, the disclosure provides a method for evaluating the sensitivity of a patient to an MET inhibitor, where the method includes a step of detecting whether a tumor sample cell contains the first nucleotide sequence, the second nucleotide sequence, the third nucleotide sequence, the fourth nucleotide sequence, the first amino acid sequence or the second amino acid sequence, and if it is detected that the tumor sample cell contains at least one of the first nucleotide sequence, the second nucleotide sequence, the third nucleotide sequence, the fourth nucleotide sequence, the first amino acid sequence or the second amino acid sequence, it is determined that the patient corresponding to the tumor sample cell are sensitive to the MET inhibitor. Where the first nucleotide sequence, the second nucleotide sequence, the third nucleotide sequence and the fourth nucleotide sequence can be detected by a method of PCR, RT-RPA, nucleic acid hybridization or high-throughput sequencing, and the first amino acid sequence and the second amino acid sequence can be detected by immunoblotting.

The disclosure is further illustrated below by the examples, but the disclosure is not thus limited.

### Preparation Example

This preparation example was used for obtaining glioblastoma PDC (patient derived cell) samples, total RNAs and total cDNAs.

Four glioblastoma samples were collected by using operations conforming to the standards of the medical ethical committee, where for each patient from whom a sample was collected, the consent of himself or herself and his or her therapist was obtained before the sample was collected, having a written proof material.

Enzymolysis was separately carried out on the four collected glioblastoma samples to obtain glioblastoma PDC samples A, B, C and D.

The total RNAs of the glioblastoma PDC samples A, B, C and D were respectively extracted by using an RNA extraction kit (purchased from Qiagen) according to its operation instruction. The extracted total RNAs were detected by an integrity analyzer, and it was confirmed that the RNA integrity number (RIN) of the total RNAs was larger than 7.0. Corresponding double-stranded cDNA was synthesized by using a reverse transcription kit (purchased from Invitrogen) according to its operation instruction and by using each total RNA as a template.

### Example 1

In this example, the total cDNAs of each glioblastoma PDC sample synthesized by the preparation example was verified.

(1) Verification of cDNA with an exon 10 deleted by using a next-generation sequencing technology.

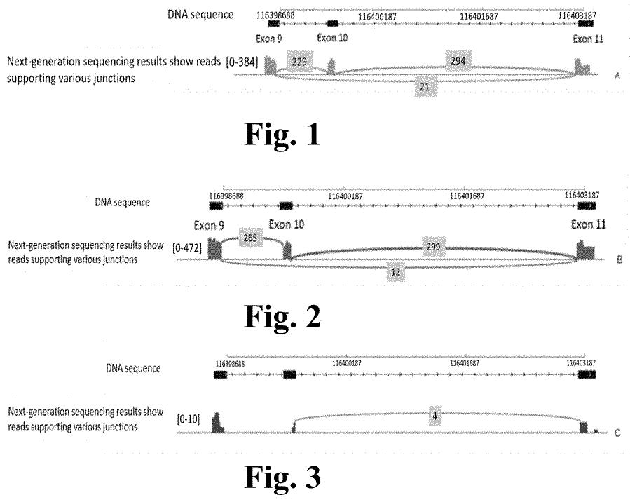

The total cDNAs of the glioblastoma PDC samples A, B, C and D were respectively sequenced by using the next-generation sequencing technology, then the number of reads supporting exons 9-10, exons 10-11 and exons 9-11 of a MET gene was identified by using IGV (Insulin Genomics Viewer) software, and if the number of reads was greater than or equal to 3, it was indicated that cDNA of the MET gene with the exon 10 deleted existed. The test results are shown in FIGS. 1-4.

Where FIG. 1 is a schematic diagram of the number of reads of exons 9-11 of the MET gene of a glioblastoma PDC sample A provided by the examples of the disclosure; FIG. 2 is a schematic diagram of the number of reads of exons 9-11 of the MET gene of a glioblastoma PDC sample B provided by the examples of the disclosure; FIG. 3 is a schematic diagram of the number of reads of exons 9-11 of the MET gene of a glioblastoma PDC sample C provided by the examples of the disclosure; and FIG. 4 is a schematic diagram of the number of reads of exons 9-11 of the MET gene of a glioblastoma PDC sample D provided by the examples of the disclosure.

It can be known from FIGS. 1-4 that the total cDNAs of the glioblastoma PDC sample A and the total cDNAs of the glioblastoma PDC sample B both had the cDNA of the MET gene with the exon 10 deleted.

(2) Verification of cDNA with an exon 19 deleted by using a next-generation sequencing technology.

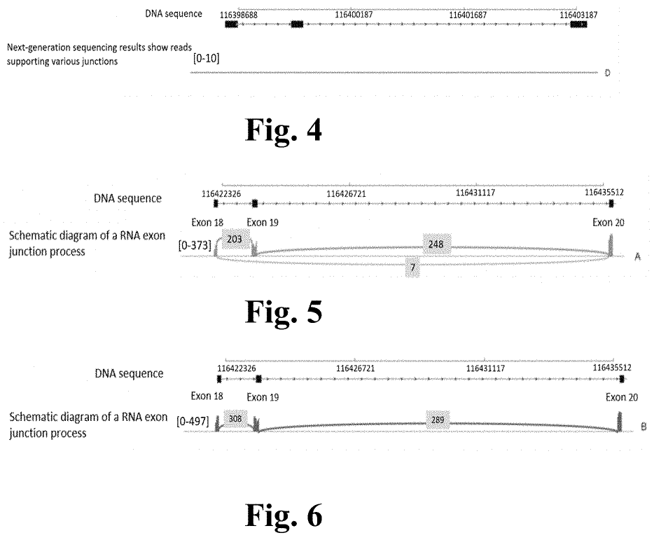

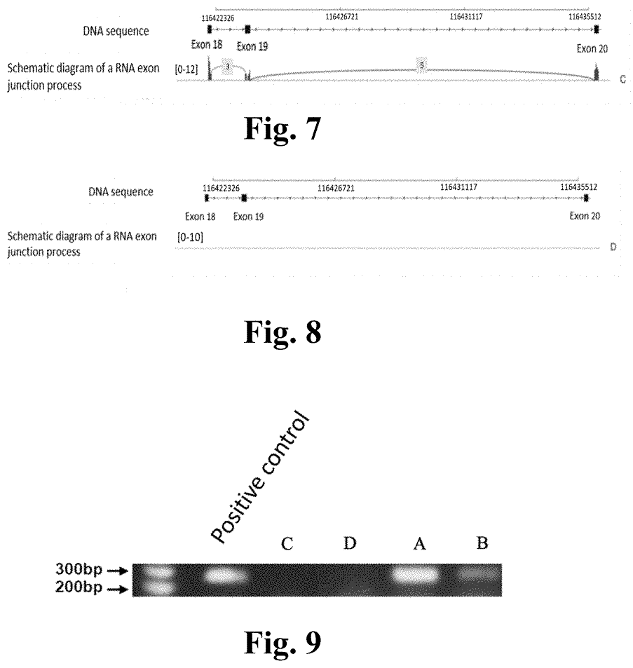

The total cDNAs of the glioblastoma PDC samples A, B, C and D were respectively sequenced by using the next-generation sequencing technology, then the number of reads supporting exons 18-19, exons 19-20 and exons 18-20 of a MET gene was identified by using IGV (Insulin Genomics Viewer) software, and if the number of reads was greater than or equal to 3, it was indicated that cDNA of the MET gene with the exon 19 deleted existed. The test results are shown in FIGS. 5-8.

Where FIG. 5 is a schematic diagram of the number of reads of exons 18-20 of the MET gene of a glioblastoma PDC sample A provided by the examples of the disclosure; FIG. 6 is a schematic diagram of the number of reads of exons 18-20 of the MET gene of a glioblastoma PDC sample B provided by the examples of the disclosure; FIG. 7 is a schematic diagram of the number of reads of exons 18-20 of the MET gene of a glioblastoma PDC sample C provided by the examples of the disclosure; and FIG. 8 is a schematic diagram of the number of reads of exons 18-20 of the MET gene of a glioblastoma PDC sample D provided by the examples of the disclosure.

It can be known from FIGS. 5-8 that the total cDNAs of the glioblastoma PDC sample A has the cDNA of the MET gene with the exon 19 deleted.

(3) Verification of cDNA with the exon 10 deleted by using a PCR (polymerase chain reaction) technology.

Primers used for PCR verification were an upstream primer as shown in SEQ ID NO. 19 and a downstream primer as shown in SEQ ID NO. 20. The PCR operation was carried out according to the instructions of synthetic primers and a PCR kit. A PCR product was subjected to agarose gel nucleic acid electrophoresis to show whether an amplification band existed or not, and the amplification result is as shown in FIG. 9.

FIG. 9 is an agarose gel electrophoretogram of PCR amplification products of glioblastoma PDC samples A, B, C and D provided by the examples of the disclosure.

It can be known from FIG. 9 that the total cDNAs of the glioblastoma PDC sample A and the total cDNAs of the glioblastoma PDC sample B both had the cDNA of the MET gene with the exon 10 deleted.

(4) Verification of cDNA with the exon 19 deleted by a PCR (polymerase chain reaction) technology.

Primers used for PCR verification were an upstream primer as shown in SEQ ID NO.

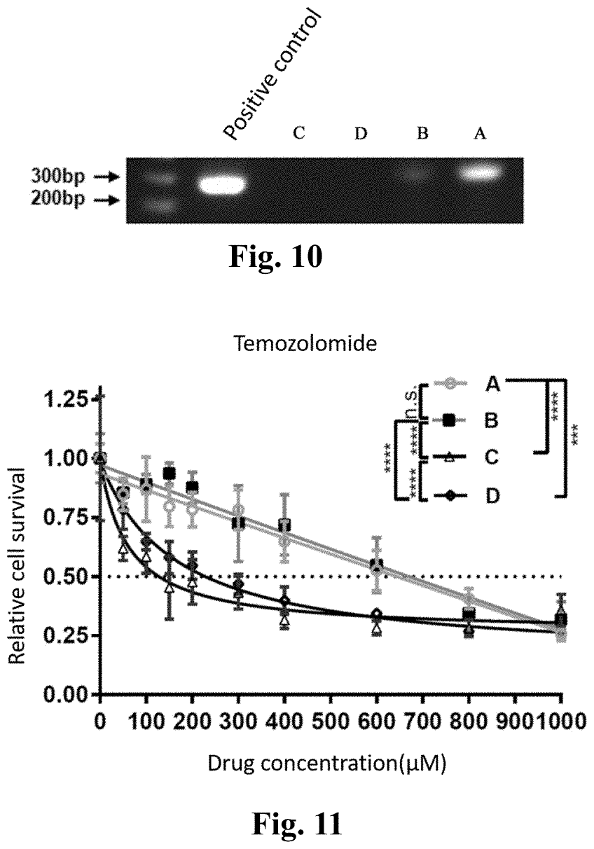

22 and a downstream primer as shown in SEQ ID NO. 23. The PCR operation was carried out according to the instructions of synthetic primers and a PCR kit. A PCR product was subjected to agarose gel nucleic acid electrophoresis to show whether an amplification band existed or not, and the amplification result is as shown in FIG. 10.

FIG. 10 is an agarose gel electrophoretogram of PCR amplification products of glioblastoma PDC samples A, B, C and D provided by the examples of the disclosure.

It can be known from FIG. 10 that the total cDNAs of the glioblastoma PDC sample A had the cDNA of the MET gene with the exon 19 deleted.

### Example 2

This example was used for verifying the sensitivity of each glioblastoma PDC sample prepared by the preparation example to an antitumor drug temozolomide (TMZ) and a MET inhibitor crizotinib.

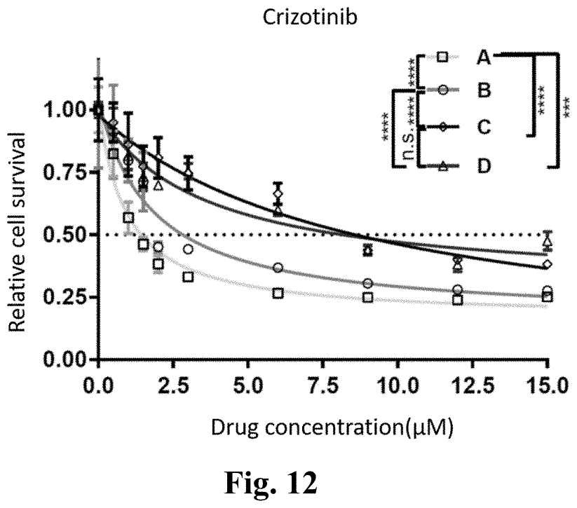

The glioblastoma PDC samples A, B, C and D were taken, re-suspended by using a special culture medium, and then respectively inoculated into a 96-well cell culture plate, so that each culture well contained 3000 cells. After inoculation was completed, the 96-well cell culture plate was placed in an incubator containing 5% CO₂ at 37° C., and incubation was performed for 24 h. After incubation was finished, TMZ or crizotinib with different concentrations were added into culture wells, and 10 replicate wells were set for each concentration gradient. After the drug was added, the incubation was continued to be performed for 72 h, then the viable count in each culture well was measured by using CCK-8, and the results are shown in Table 1, Table 2, FIG. 11 and FIG. 12.

TABLE 1
Viable count after treatment with different concentrations of TMZ

| | | Living cell |
|---|---|---|
| Glioblastoma | TMZ | proportion (a value |
| PDC sample | concentration, μM | relative to CCK8) |
| A | 0 | 1 |
| | 50 | 0.848085 |
| | 100 | 0.869555 |
| | 150 | 0.796761 |
| | 200 | 0.784317 |
| | 300 | 0.782419 |
| | 400 | 0.648194 |
| | 600 | 0.526062 |
| | 800 | 0.405245 |
| | 1000 | 0.257712 |
| B | 0 | 1 |
| | 50 | 0.854895 |
| | 100 | 0.890512 |
| | 150 | 0.936898 |
| | 200 | 0.876325 |
| | 300 | 0.724021 |
| | 400 | 0.718298 |
| | 600 | 0.548946 |
| | 800 | 0.34503 |
| | 1000 | 0.317545 |
| C | 0 | 1 |
| | 50 | 0.618833 |
| | 100 | 0.584119 |
| | 150 | 0.453601 |
| | 200 | 0.476835 |
| | 300 | 0.431461 |
| | 400 | 0.316387 |
| | 600 | 0.281673 |
| | 800 | 0.28345 |
| | 1000 | 0.35848 |
| D | 0 | 1 |
| | 50 | 0.782796 |
| | 100 | 0.647003 |
| | 150 | 0.579895 |
| | 200 | 0.546755 |
| | 300 | 0.46814 |
| | 400 | 0.398432 |
| | 600 | 0.343539 |
| | 800 | 0.268393 |
| | 1000 | 0.252906 |

TABLE 2
Viable count after treatment with different
concentrations of crizotinib

| | | Living cell |
|---|---|---|
| Glioblastoma | Crizotinib | proportion (a value |
| PDC sample | concentration, μM | relative to CCK8) |
| A | 0 | 1 |
| | 0.5 | 0.824008 |
| | 1 | 0.568422 |
| | 1.5 | 0.462403 |
| | 2 | 0.383442 |
| | 3 | 0.331603 |
| | 6 | 0.266346 |
| | 9 | 0.248212 |
| | 12 | 0.238686 |
| | 15 | 0.25182 |
| B | 0 | 1 |
| | 0.5 | 0.912756 |
| | 1 | 0.797768 |
| | 1.5 | 0.712466 |
| | 2 | 0.451969 |
| | 3 | 0.44294 |
| | 6 | 0.368899 |
| | 9 | 0.305861 |
| | 12 | 0.280566 |
| | 15 | 0.276901 |
| C | 0 | 1 |
| | 0.5 | 0.949599 |
| | 1 | 0.86187 |
| | 1.5 | 0.773808 |
| | 2 | 0.808238 |
| | 3 | 0.746509 |
| | 6 | 0.664314 |
| | 9 | 0.438308 |
| | 12 | 0.400312 |
| | 15 | 0.382373 |
| D | 0 | 1 |
| | 0.5 | 0.924667 |
| | 1 | 0.816214 |
| | 1.5 | 0.732671 |
| | 2 | 0.698619 |
| | 3 | 0.754408 |
| | 6 | 0.599543 |
| | 9 | 0.438101 |
| | 12 | 0.381239 |
| | 15 | 0.475231 |

It can be seen from Table 1 and FIG. 11 that the glioblastoma PDC samples A and B were insensitive to the anti-tumor drug temozolomide (TMZ), and the glioblastoma PDC samples C and D were sensitive to the anti-tumor drug temozolomide (TMZ).

It can be seen from Table 2 and FIG. 12 that the glioblastoma PDC samples A, B, C and D were sensitive to the MET inhibitor crizotinib; moreover, the sensitivity of the glioblastoma PDC samples A and B to crizotinib were higher than that of the glioblastoma PDC samples C and D; in addition, the sensitivity of the glioblastoma PDC sample A to crizotinib was higher than that of the glioblastoma PDC sample B.

Thus it can be seen that when the deletion of the exon 10 and the deletion of the exon 19 of the MET gene existed in the tumor cells, the tumor cells had high sensitivity to the MET inhibitor, and when the deletion of the exon 10 and the deletion of the exon 19 of the MET gene existed in the tumor cells at the same time, the sensitivity of the tumor cells to the MET inhibitor was the highest.

The preferred examples of the disclosure are described in detail above in combination with the drawings, however, the disclosure is not limited to the specific details in the above examples, in the technical concept range of the disclosure, the technical solution of the disclosure can be subjected to various simple variations, and these simple variations all belong to the protection range of the disclosure.

In addition, it should be noted that the specific technical features described in the specific examples can be combined in any appropriate mode without contradiction, and in order to avoid unnecessary repetition, various possible combination modes are not described any more in the disclosure.

In addition, various different examples of the disclosure can also be combined at will, and as long as the examples do not violate the idea of the disclosure, the examples also should be regarded as the contents disclosed by the disclosure.

1. A kit for evaluating the sensitivity of a patient to an MET inhibitor, wherein the kit comprises a first primer pair capable of specifically amplifying a first nucleotide sequence, and/or a first probe capable of specifically binding to the first nucleotide sequence, wherein the first nucleotide sequence is shown as SEQ ID NO. 1; and/or,
the kit comprises a second primer pair capable of specifically amplifying a second nucleotide sequence, and/or a second probe capable of specifically binding to the second nucleotide sequence, wherein the second nucleotide sequence is shown as SEQ ID NO. 4.

2. The kit according to claim 1, wherein the first nucleotide sequence is as shown in SEQ ID NO. 2; preferably, the first nucleotide sequence is as shown in SEQ ID NO. 3, wherein the nucleotide sequence as shown in SEQ ID NO. 3 is a cDNA sequence of the MET gene with an exon 10 deleted; and
the second nucleotide sequence is as shown in SEQ ID NO. 5; preferably, the second nucleotide sequence is as shown in SEQ ID NO. 6, wherein the nucleotide sequence as shown in SEQ ID NO. 6 is a cDNA sequence of the MET gene with an exon 19 deleted.

3. The kit according to claim 1, wherein the patient is a glioma patient.

4. A kit for evaluating the sensitivity of a patient to an MET inhibitor, wherein the kit comprises a third primer pair capable of specifically amplifying a third nucleotide sequence, and/or a third probe capable of specifically binding to the third nucleotide sequence, wherein the third nucleotide sequence is shown as SEQ ID NO. 7; and/or,
the kit comprises a fourth primer pair capable of specifically amplifying a fourth nucleotide sequence, and/or a fourth probe capable of specifically binding to the fourth nucleotide sequence, wherein the fourth nucleotide sequence is shown as SEQ ID NO. 10.

5. The kit according to claim 4, wherein the third nucleotide sequence is as shown in SEQ ID NO. 8; preferably, the third nucleotide sequence is as shown in SEQ ID NO. 9, wherein the nucleotide sequence as shown in SEQ ID NO. 9 is an mRNA sequence of the MET gene with an exon 10 deleted; and
the fourth nucleotide sequence is as shown in SEQ ID NO. 11; preferably, the fourth nucleotide sequence is as shown in SEQ ID NO. 12, wherein the nucleotide sequence as shown in SEQ ID NO. 12 is an mRNA sequence of the MET gene with an exon 19 deleted.

6. A kit for evaluating the sensitivity of a patient to an MET inhibitor, wherein the kit comprises a first antibody for resisting a first amino acid sequence, wherein the first amino acid sequence is shown as SEQ ID NO. 13; and/or,
the kit comprises a second antibody for resisting a second amino acid sequence, wherein the second amino acid sequence is shown as SEQ ID NO. 16.

7. The kit according to claim 6, wherein the first amino acid sequence is as shown in SEQ ID NO. 14; preferably, the first amino acid sequence is as shown in SEQ ID NO. 15, wherein the amino acid sequence as shown in SEQ ID NO. 15 is encoded by mRNA as shown in SEQ ID NO. 9; and
the second amino acid sequence is as shown in SEQ ID NO. 17; preferably, the second amino acid sequence is as shown in SEQ ID NO. 18, wherein the amino acid sequence as shown in SEQ ID NO. 18 is encoded by mRNA as shown in SEQ ID NO. 12.

8. (canceled)

9. (canceled)

10. (canceled)

11. The kit according to claim 2, wherein the patient is a glioma patient.
